(19) United States
(12) Patent Application Publication
(10) Pub. No.: US 2017/0100131 A1
(43) Pub. Date: Apr. 13, 2017

(54) Flywheel Tourniquet System and Method of Application

(71) Applicants: Justin Leo Olbu, Issaquah, WA (US); Devin William Kim, Anchorage, AK (US)

(72) Inventors: Justin Leo Olbu, Issaquah, WA (US); Devin William Kim, Anchorage, AK (US)

(21) Appl. No.: 15/284,548

(22) Filed: Oct. 4, 2016

Related U.S. Application Data
(60) Provisional application No. 62/239,842, filed on Oct. 9, 2015.

(51) Int. Cl. A61B 17/132

(52) U.S. Cl. CPC A61B 17/1327; A61B 2017/12004

(57) ABSTRACT

A system and method for application of a tourniquet system utilizing a strap capable of tightening annularly about the limb of a user, a buckling component and a tightening mechanism to quickly tighten the buckled strap about the limb of an injured entity.

### CROSS-REFERENCE TO RELATED APPLICATIONS

This application claims priority of U.S. provisional application 62/239,842, filed Oct. 9, 2015.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISK APPENDIX

Not Applicable

### FIELD OF THE INVENTION

The present invention is in the field of emergency medicine. More specifically, the present invention is in the technical field of first aid. Most specifically, the present invention is in the technical field of halting limb hemorrhaging.

### BACKGROUND OF THE INVENTION

The current standard means to halt limb hemorrhaging is to wrap a length of flexible material around the limb of an injured patient at a locus approximately four to five inches above the wound. Once the material is wrapped around the patient's limb, a rod upon the flexible material is rotated about the limb, tightening the flexible material. Doing so causes the material to tighten annularly around the limb, constricting the blood vessels and causing the bleeding to stop. Once blood flow is stopped, the rod is secured to prevent release while the patient is transported to a hospital or other location able to provide additional medical attention.

While tourniquet systems found in the prior art are useful, inherent design problems become readily apparent when used in emergency situations. Many present systems require threading of straps through buckles or the like, which may not be difficult in practice, but may difficult in the real life scenarios. For instance, bleeding may make threading difficult. Or, heightened fear, anxiety, adrenaline levels and/or stress may make application of available systems difficult. Furthermore, these factors, along with blood loss and shock may make available systems excessively difficult for self-application.

Therefore, there is a need for a system that would allow application of a tourniquet quickly in a variety of environmental conditions, by users with varying degrees of medical training (including none). The goal of the present invention is to remedy the deficiencies found in the prior art.

The inventor has performed a search of the prior art and believes the present invention is a new and useful invention for which patent protection is warranted.

### SUMMARY OF THE INVENTION

Within the preferred embodiment, and any alternative embodiments, a tourniquet system utilizing a flywheel to assist in application is provided. The system is comprised of a strap assembly, means for tightening said strap assembly, and a means for releasing the strap.

The present invention would allow for self-application, or for application onto another by an individual with little to no formal medical training, in a variety of conditions.

### BRIEF DESCRIPTION OF THE DRAWINGS

FIG. 1 is an exploded view of the preferred embodiment of the present invention;

FIG. 2 is a flowchart outlining the steps of application of the tourniquet.

### DETAILED DESCRIPTION OF THE INVENTION

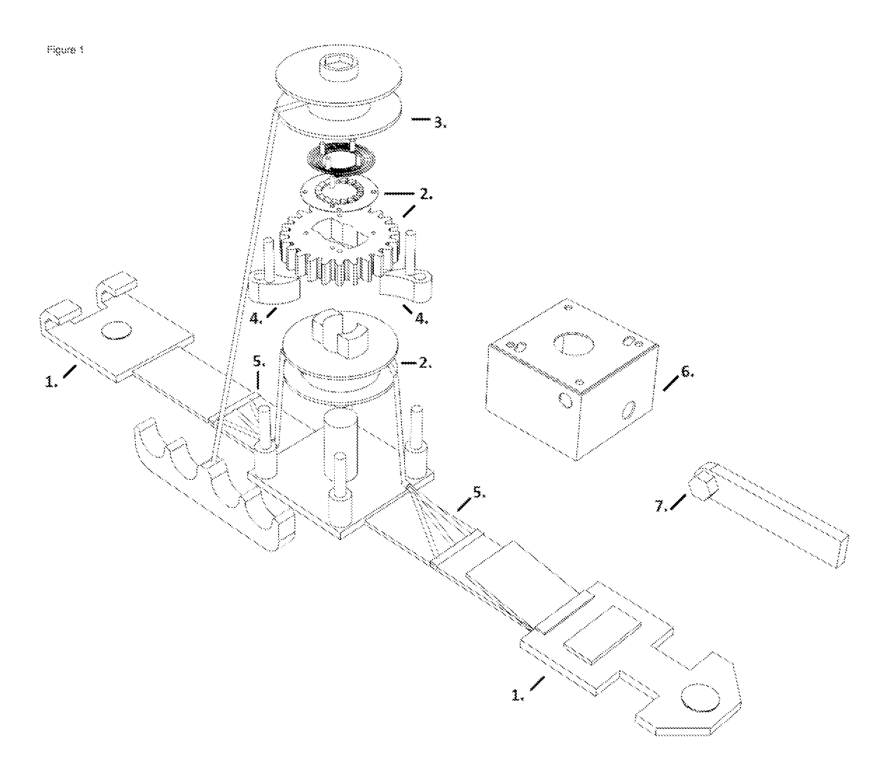

Referring now to the exploded view of the present invention as shown in FIG. 1, there is shown the buckle assembly 1. The preferred embodiment of the present invention will use a magnetic buckle system 1 to guide and couple both ends of the buckle assembly 1 at the terminal ends of the strap and strap wires 5. An outer casing 6 rests atop the strap and strap wires 5. Within the enclosure is featured a spool assembly with a locking plate 2. The spool assembly and locking plate 2 mates with wires along the strap 5, so that a rotational force applied to the spool assembly and locking plate 2 causes the strap wires 5 to tighten and constrict the strap 5 about the limb of the user. The preferred embodiment of the present invention will use a flywheel assembly 3 with a locking mechanism 3, pull cord 3 and cord retraction spring 3 to apply rotational force to the spool assembly and locking plate 2. A ratcheting gear lock 4 and assist wrench 7 may also be featured to further tighten or loosen the entire system about the limb of the injured user.

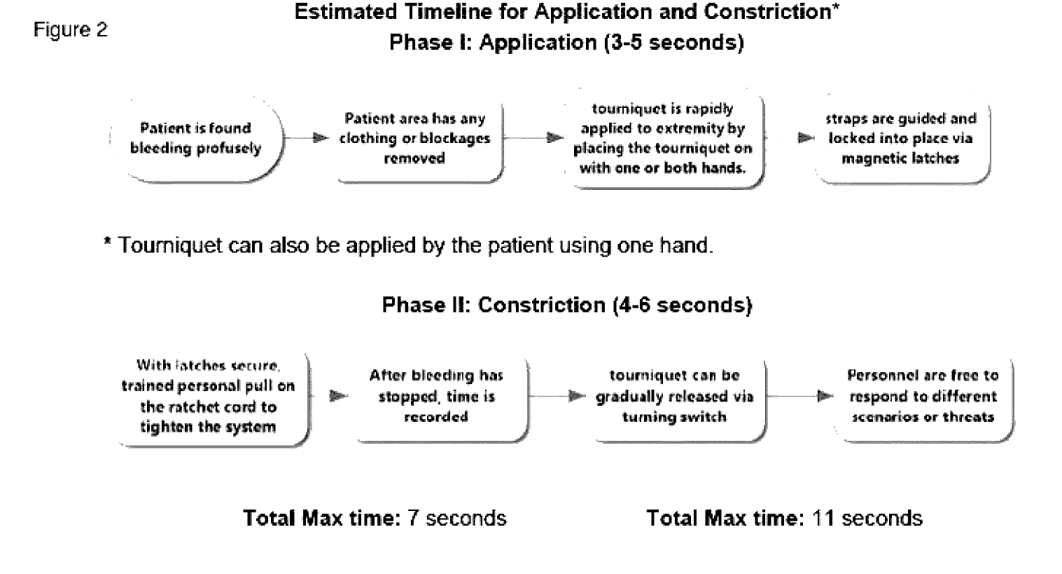

FIG. 2 outlines the steps that would be taken to apply the disclosed system and method of application.

Referring now to both FIGS. 1 and 2, the system would be applied to an injured user as follows: the strap 5 would be wrapped above the site of the injury. The buckle assemble 1 would then be used to fasten the present invention about the user. Pulling on the handle of the flywheel cord 3 in a manner similar to pulling on the starter cord of a standard lawnmower would apply a rotational force to the spool assembly and locking plate 2. This rotational force in turn would cause wires located along the strap 5 to wind about the spool assembly and locking plate 2. Once the desired level of constriction is reached, releasing the flywheel cord 3, would return the flywheel cord 3 to within the outer casing 6 by means of tension on the flywheel spring 3. If further tightening is desired, use of an assist wrench 7 would enable a user to apply additional rotational force to the spool assembly and locking plate. Conversely, an assist wrench 7 could be used to apply rotational force in an opposing direction to loosen the wires and strap 5 if loosening or removal is desired.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the described embodiment, method, and examples, but by all embodiments and methods within the scope and spirit of the invention as claimed.

What is claimed is:

1. A tourniquet system comprising:
a strap with a means for coupling the terminal ends of the strap and a wire running horizontally along the strap to tighten the strap;
a means for tightening the strap annularly about the limb of a user, said means affixed along the body of the strap between the terminal ends of the strap and utilizing a spool to tighten the wire along the strap.

2. The system of claim 1, wherein the means for coupling the terminal ends of the strap is a magnetic buckle, to allow quick coupling of the terminal ends of the strap.

3. The system of claim 1, wherein the means for tightening the strap is a flywheel capable of turning a spool to gather the wire along the strap.

4. The system of claim 1, wherein a secondary ratcheting gear may be used to further tighten the tourniquet about the limb of the user, or assist in removal if removal is desired.

5. A tourniquet system consisting of:
a strap with a means for coupling the terminal ends of the strap, a wire running horizontally along the strap between the terminal ends and along the body of the strap a means for tightening the wire about a spool, said means featured along the body of the strap and in line with the wire.

6. The system of claim 5, wherein the means for coupling the terminal ends of the strap is a magnetic buckle, to allow quick coupling of the terminal ends of the strap.

7. The system of claim 5, wherein a flywheel is utilized to apply rotational force to the spool to gather the wire about the body of the spool to tighten the strap about the limb of a user.

8. The system of claim 5, wherein a secondary ratcheting gear may be used to further tighten the tourniquet about the limb of the user, or assist in removal if removal is desired.
